(19) United States
(12) Patent Application Publication
(10) Pub. No.: US 2014/0228735 A1
(43) Pub. Date: Aug. 14, 2014

(54) DELIVERY DEVICE WITH INVERTIBLE DIAPHRAGM

(71) Applicant: TRISTAR WELLNESS SOLUTIONS, INC., Westport, CT (US)

(72) Inventor: Matthew W. Hoskins, Bend, OR (US)

(73) Assignee: TRISTAR WELLNESS SOLUTIONS, INC., Westport, CT (US)

(21) Appl. No.: 14/252,977

(22) Filed: Apr. 15, 2014

Related U.S. Application Data
(63) Continuation of application No. 12/946,671, filed on Nov. 15, 2010, now abandoned.
Application No. 12/946,671 is a continuation-in-part of application No. PCT/US2009/044400, filed on May 18, 2009.
(60) Provisional application No. 61/071,766, filed on May 16, 2008.

(51) Int. Cl. A61F 13/26

(52) U.S. Cl. CPC A61F 13/26
USPC 604/14

(57) ABSTRACT

A method and device are disclosed for delivering items into the body of a subject. The device is elongated, and includes a flexible diaphragm having a proximal face and a distal face. The diaphragm is inverted into the device to form a receptacle that holds an item to be delivered. Fluid pressure is applied against the proximal face of the flexible diaphragm to deliver the item from the receptacle and out of the delivery device into the body. In one embodiment, the device is a one-piece device having compressible walls. The diaphragm, which may be a portion of the chamber wall, invaginates within the chamber to form the receptacle. Compression of the flexible walls of the device increases pressure on a proximal face of the diaphragm to evaginate the diaphragm out of the chamber and deliver an item contained in the chamber.

### CROSS REFERENCE TO RELATED APPLICATION

This is a Continuation of U.S. patent application Ser. No. 12/946,671 filed Nov. 15, 2010, which is a Continuation-in-part (CIP) of PCT Application No. PCT/US2009/044400, filed May 18, 2009, which claims the benefit of U.S. Provisional Application No. 61/071,766, filed May 16, 2008. The CIP, PCT and provisional applications are all incorporated herein in their entirety.

### FIELD

The invention relates to methods and devices for introducing items (such as medical or hygienic products) into a body cavity.

### BACKGROUND

Formed items (such as tampons or medical implants) are sometimes delivered into the body for hygienic, therapeutic or diagnostic purposes. Such items are often indwelling devices that are left in situ for short-term or long-term periods of time. The tools and methods of introduction depend on the type and size of item being placed and the access route to the placement location. Indwelling items may be placed directly by hand into a body orifice or surgical opening, but internal delivery is typically achieved using a delivery tool that facilitates placement. Delivery tools can enhance the placement and function of indwelling devices by providing lower-profile device introduction, device protection during insertion, improved access capability, and controlled, ergonomic deployment. Indwelling treatment aids that can be used with delivery tools include menstrual tampons, suppositories, liquid preparations (such as solutions or dispersions) and other pharmaceuticals (such as solid drug delivery forms), drug release devices, stents, vena-cava filters, radiation sources (such as radioactive seeds or implants), embolization devices (such as expandable coils for neurovascular embolization), and fallopian tube plugs, among others.

Elongated tubes or syringes have previously been used to deliver items to a target site in the body. The tip of the device is positioned at the desired location within the body and an internal pusher is advanced within the elongated tube or syringe to physically contact the item and push it out of the tube. This approach has certain disadvantages, such as requiring use of a long conduit, and the pusher member may not have sufficient column strength to reliably eject the item to be delivered. In other situations, the rigidity required of the device imposes design restrictions on material properties. Another disadvantage of this approach is that the item may be misdirected as it is ejected from the end of the device. The ejected item also meets with strong lateral pressure as it is deployed into a restricted space. Frictional forces can also hamper expulsion or induce trauma as the indwelling item is pushed from the delivery tube. In certain applications, the ergonomics of the device are uncomfortable for the operator.

An alternative delivery system places the item in a receptacle and unsheathes the item by retracting an elongated tube from around the item while holding the indwelling item stationary by supporting it with an internal member. This method avoids the potential for misdirection and frictional resistance associated with ejection of the indwelling item out of the tip of the elongated tube. However, unsheathing the item can be cumbersome because the internal member must be held stationary while retracting the sheath. Misplacement can occur if the operator inadvertently advances the internal member or moves the entire assembly during unsheathing.

Menstrual tampon applicators are a common delivery device in which an outer barrel holds the tampon within its proximal portion, and an internal plunger advances the tampon out of the barrel. In use, the tampon applicator is grasped by the fingers and the barrel portion is inserted 1-2 inches into the vagina. A spare finger or second hand is then used to push the tampon plunger inward to expel the tampon. Modern tampon applicators are typically made of plastic and feature smooth, low-friction surfaces and rounded, petal-formed ends to ease insertion and increase comfort. Despite the sophistication of these designs, they can still be difficult to use. Insertion of the applicator can be difficult if there is not enough lubricity between the vaginal walls and the applicator. The petal segments on the applicator tip can entangle hair or abrade tissue during insertion, deployment, and withdrawal of the applicator. Tampon advancement out of the applicator barrel may be difficult due to frictional resistance with the collapsed vaginal walls. Maintaining a grip on the slender applicator during insertion and expulsion can also be a challenge, and plunger advancement can be awkward and sometimes requires the use of both hands.

Disposal of plastic applicators can be problematic. Although plastic applicators are often preferred over cardboard models due to their smoothness and functionality, the plastic applicators contribute to landfill volume and do not degrade as readily as flushable cardboard models. It would be advantageous to have a plastic tampon applicator that is either biodegradable or water dispersible, and that also overcomes the ergonomic and functional disadvantages of current tampon applicators.

### SUMMARY

A device is disclosed herein for delivering an item to a delivery site within the body of a subject. The device is elongated and has a proximal end and a distal end. The device further includes a flexible diaphragm having a proximal face and a distal face, and the diaphragm is invertible to form an invaginated receptacle that holds an item to be delivered into the body. The flexible diaphragm moves in response to fluid pressure against its proximal face to evaginate the diaphragm and deliver the item from the receptacle and out of the delivery device into the body.

In particular embodiments, the delivery device forms a hand-held compressible chamber capable of internal pressurization, and the flexible diaphragm forms a portion of the distal end of the chamber. The flexible diaphragm is inverted to form the invaginated receptacle within the chamber. The chamber of the delivery device may have an enlarged proximal portion with a larger diameter than the distal portion. The enlarged proximal portion provides a compressible handgrip for increasing pressure within the chamber when the handgrip is compressed to thereby apply the fluid pressure to the proximal face of the flexible diaphragm. In particular embodiments, the handgrip in inclined relative to the distal portion of the delivery device to improve the ergonomics of the device and facilitate its manipulation by a user.

The fluid pressure that is applied to the proximal face of the diaphragm may be pressure from a liquid or gas, but gas pressure is used in several disclosed embodiments. The chamber may be a self-contained, substantially sealed or unitary chamber that contains a sufficient amount of a fluid (such as a gas or liquid) to apply evagination pressure against the flexible diaphragm when the chamber is compressed. Alternatively, the chamber communicates with an external pressurization device, such as a syringe or compressible bulb that is in fluid connection with the chamber.

In another disclosed embodiment, the delivery device is a tube having an open distal end from which the item is delivered, and the flexible diaphragm flexes within the tube. The diaphragm may be secured, for example, around its edges to a wall of the tube, and the diaphragm is elongated and sufficiently greater in area than a diameter of the tube that the diaphragm folds against the walls of the tube to invert and form the receptacle. Fluid pressure applied against the proximal face of the diaphragm at least partially evaginates the diaphragm. The diaphragm may be secured to the internal wall of the tube sufficiently near the open distal tip of the tube that at least a portion of the diaphragm protrudes out of the open distal end of the tube when the diaphragm evaginates.

In certain embodiments, the delivery device is used to introduce an item into a location that is not readily accessible from a surface orifice of the body. In such an example, the delivery device may be attached to an access tube having a pressure lumen that communicates with the diaphragm, and pressure is introduced into the pressure lumen to evaginate the diaphragm and deliver the item into the body. The access tube may be a flexible catheter that is introduced into a vascular structure or other non-vascular organ cavity or lumen within the body.

In other disclosed embodiments, the inverted diaphragm is elongated and/or forms a substantially cylindrical receptacle. The item to be delivered may be positioned within the receptacle. In a particularly disclosed embodiment, the item is a tampon for introduction into the vagina.

Also disclosed herein is a method of delivering an item into the body of a subject by introducing the delivery device into a target location within the body where the item is to be delivered. For example, to deliver a tampon to the vagina, the distal end of the delivery device is introduced through the introitus into the vagina. Pressure is then applied against the proximal face of the flexible diaphragm to evaginate the diaphragm and deliver the item (such as the tampon) from the receptacle and out of the delivery device into the body. The device is then withdrawn from the body.

In one embodiment the delivery device forms a flexible and/or compressible chamber capable of internal pressurization, and the flexible diaphragm is a portion of the distal end of the chamber. The flexible diaphragm, which is invaginated within the chamber to form the receptacle for the item, is at least partially evaginated by increasing fluid pressure within the chamber to deliver the item from the receptacle into the body. For embodiments in which the compressible chamber of the delivery device has an enlarged proximal handgrip portion, the fluid pressure is increased by squeezing the handgrip. The chamber may be a self-contained, substantially sealed enclosure that contains a gas, and gas pressure is applied against the flexible diaphragm by compressing the chamber to increase pressure within the chamber. Alternatively, when the chamber communicates with an external pressurization device such as a syringe or compression bulb, fluid pressure is applied against the diaphragm by introducing fluid from the pressurization device into the chamber to increase pressure within the chamber to evaginate the flexible diaphragm.

For tube-shaped embodiments of the delivery device, the diaphragm may be secured around the edges of the diaphragm to a wall of the tube. The diaphragm is sufficiently greater in area than a diameter of the tube that the diaphragm folds against the walls of the tube to invert and form the receptacle, and the fluid pressure is applied against the proximal face of the diaphragm through the tube to at least partially evaginate the diaphragm. In some embodiments, the evaginated diaphragm protrudes at least partially out of the open distal end of the tube.

The foregoing and other objects, features, and advantages of the invention will become more apparent from the following detailed description, which proceeds with reference to the accompanying figures.

### BRIEF DESCRIPTION OF THE DRAWINGS

FIG. 1A is a fragmentary view of an access catheter with a delivery device on its distal tip. FIG. 1B is an enlarged view of the delivery device with the diaphragm fully invaginated, while FIG. 1C illustrates partial evagination of the diaphragm.

FIGS. 2A, 2B and 2C are a series of enlarged fragmentary side views, partially in phantom, of the catheter and delivery device shown in FIG. 1, illustrating evagination of the flexible membrane to deliver a formed item from within the receptacle.

FIGS. 2D, 2E and 2F show an embodiment of the device in which the catheter is guided by a monorail wire.

FIGS. 3A, 3B and 3C are views similar to FIGS. 2A, 2B and 2C, but illustrating delivery of an item which is secured to the flexible diaphragm but detaches as the diaphragm evaginates.

FIGS. 4A, 4B and 4C are a series of schematic side views partially in phantom that illustrate delivery of an item from a self-contained version of the delivery device in which the delivery device is a self-contained, substantially sealed chamber in which the distal round-ended portion of the device is invaginated into the chamber to form the receptacle. As fluid pressure in the chamber is increased, the receptacle evaginates to dispel the item from the receptacle.

FIG. 5 is a schematic drawing of another embodiment of the device, similar to FIGS. 4A, 4B and 4C, but in which a compressible bulb provides an external pressurization device for the chamber of the delivery device.

FIGS. 6A, 6B and 6C illustrate an embodiment of the delivery device that is adapted for ergonomic use as an applicator for an absorbent material such as a tampon.

FIGS. 7A and 7B illustrate an alternative embodiment of the device shown in FIGS. 6A and 6B, wherein petalous projections partially close the distal open end of the device to provide a cap through which the flexible diaphragm is evaginated.

FIGS. 8A, 8B and 8C are schematic drawings of an embodiment similar to FIG. 7, but the petalous projections are contained within the flexible diaphragm.

FIGS. 9A and 9B are schematic views that illustrate an embodiment of the tampon applicator in which the flexible diaphragm has a secondary invagination in which the tampon string is placed.

FIGS. 10A and 10B illustrate a tampon delivery system similar to FIG. 9, but the string is lightly attached to the tampon receptacle.

FIGS. 11A and 11B show a tampon delivery system with a sealed, pre-pressurized chamber for facilitating tampon unloading.

FIGS. 12A and 12B illustrate another embodiment of the delivery system having a resilient frustoconical-shaped dispenser body with an air outlet/inlet opening in its wall that permits compression/re-expansion of the dispenser.

### DETAILED DESCRIPTION

### Terms

As used herein, the term “invert” refers to turning outside in, for example a diaphragm that is folded from outside a delivery device into the device. The term “evert” refers to turning inside out, for example moving from inside the delivery device toward the outside of it. The term “invaginate” refers to folding inward, for example from outside a delivery device into the device. The term “evaginate” refers to folding inside out, for example from inside a delivery device toward the outside of it.

As used herein, the singular forms “a,” “an,” and “the” refer to one or more than one, unless the context clearly dictates otherwise. The term “includes” means “comprises.” For example, a device that includes or comprises A and B contains A and B but may optionally contain C or other components other than A and B. A device that includes or comprises A or B may contain A or B or A and B, and optionally one or more other components such as C.

### General Embodiments

A delivery device is disclosed that offers ease of use and improved placement capability while reducing tissue trauma. In particular embodiments, the device is suitable for placement of catamenial products such as menstrual tampons. Certain embodiments of the device are also of small size, have less discard volume, and are more environmentally-compatible than many prior applicators.

Particular embodiments of the device deliver an indwelling item from a thin-walled elongated member capable of holding pressure. The delivery device includes a chamber made of a flexible material that is capable of folding on itself to turn outside in (invert) to form a receptacle or inside out (evert) to empty the contents of the receptacle. In such an embodiment, the portion of the device that folds in (a diaphragm) is made of the same material as the walls of the surrounding chamber, or has the same or similar degree of flexibility as the surrounding chamber. A portion (such as a tip) of the delivery device is partially or fully folded in on itself to form a receptacle of sufficient size to hold the item to be delivered. The item to be delivered (such as an indwelling object) is housed within the receptacle formed by the inverted portion. To deploy the indwelling object, the delivery device is internally pressurized. When sufficient internal pressure is generated, the inverted portion of the delivery device partially or completely unfolds from its inverted position and expels the indwelling object from the receptacle to a target site within the body.

In one embodiment of the invention a proximal open end of the delivery device is a flexible diaphragm that is sealed circumferentially to the distal end of an access tube, such as a flexible catheter. The access tube is of sufficient size and length to access a bodily orifice or vessel leading to the location where the indwelling object is to be placed. A distal end of the delivery device is closed by the flexible diaphragm, which can be folded inward on itself. The diaphragm is of sufficient length that is can be inverted only within itself, so that the inverted portion of the diaphragm is contained within a non-inverted portion of the diaphragm that surrounds it. However with additional inversion the diaphragm may be folded partially or completely into the delivery tube with the inverted portion of the diaphragm proximal to the distal portion of the diaphragm that is circumferentially sealed to the access tube. The item to be delivered is housed within the inverted portion of the diaphragm, which depending on the degree of inversion, may be inside the delivery tube. Internal pressurization of the access tube causes the inverted portion of the delivery device to evaginate, which reduces or eliminates the receptacle, and dispenses the item from the delivery device.

In some embodiments, the access tube includes a pressurization lumen that is in communication with the sealed delivery device; the pressurization lumen may be connected to a pressure source, such as a syringe. The delivery device is pre-loaded with the indwelling object housed within the receptacle. The delivery device is inserted through the appropriate bodily conduit until the delivery location is reached. The delivery tube pressurization channel is pressurized causing controlled advancement and unsheathing of the indwelling object from the delivery device. Once the indwelling object has been delivered from the receptacle, the delivery device can be withdrawn from the body.

In an alternative embodiment similar to that described above, the object to be delivered is sealed to or otherwise reversibly attached to the diaphragm that forms the receptacle. When the receptacle is inverted, the delivery object is sheathed within the inverted portion of the diaphragm, and with enough inversion is drawn into the delivery tube. When fluid pressure is applied to the proximal face of the diaphragm, the inverted portion unfolds to evaginate and dispel the object from the receptacle. As the receptacle evaginates, the unfolding action and/or pressurization disrupts the sealed connection between the delivery object and second end of the elongated member, which releases the object from the delivery device.

In another embodiment the delivery device is a thin-walled self-contained member that can be configured to work as a stand-alone delivery tool. The walls of the device form a fluid-tight chamber containing a degree of internal pressure. The dimensions of the device are appropriate for accessing a targeted bodily opening, such as the vagina through the introitus. The device has a distal portion for insertion into the body and a proximal portion that is grasped and retained outside the body. The internal pressure level and air or fluid volume within the self-contained member is set so that the wall of the distal portion can be inverted to fully sheath the item to be delivered, while providing sufficient column strength for insertion into a selected bodily orifice. The delivery device is pre-loaded with the item housed within a receptacle formed by the inverted tip. The delivery device is inserted into a bodily opening and advanced towards the target location. When the distal end of the delivery device is near the target location, advancement is discontinued, and deployment begins.

Deployment is triggered by increasing the internal pressure within the chamber of the delivery device. The increase in pressure at the distal end causes evagination of the inverted tip. As evagination progresses, the item is carried forward and gradually unsheathed until it is fully released from the delivery device. Chamber pressurization can be achieved by applying a squeezing force to the proximal portion of the delivery device. In this case, the proximal portion is sized so that it is ergonomic to squeeze and so that it contains sufficient air volume for full deployment of the item. Alternatively, pressurization may be achieved by connecting the chamber to a secondary pressure source via an air-tight connection. Examples of suitable pressurization sources are a syringe, squeeze bulb, or bladder. Alternatively the pressure source can be an electric pump, or a pre-pressurized reservoir. The pressure source can be a separate component or integrated into the delivery device.

Various design features can be integrated into the delivery device for enhancing bodily access and deployment. These include, among others, channels for tracking over a guidewire, tip deflectability, lubricious coatings, and combination with a balloon catheter. Depending on the application, other design enhancements may be desirable. These could include specially-shaped tips that facilitate access without hampering deployment, designs that integrate an access catheter with the item to be delivered (such as a stent that is deployed via pressurization and eversion from a catheter), and designs wherein the item to be delivered is hermetically sealed within the delivery device until deployment.

In a more specific embodiment, the invention is configured as a menstrual tampon delivery device. For example, the tampon applicator has a thin-walled, air-tight, pressurized chamber made of a biocompatible material and featuring distal and proximal portions. The inverted distal end of the chamber sheaths a tampon, while the proximal portion is used as a handle and squeeze bulb to pressurize the applicator chamber and cause eversion of the distal tip and tampon deployment. The distal portion of the chamber is elongated and slightly larger than a tampon in diameter. The proximal portion is sized and shaped to act as a handle to facilitate vaginal insertion. The distal end of the elongated member is folded-in on itself to create a space to accommodate a tampon and tampon string, which is loaded into this space during manufacturing. The applicator chamber's pressure level is set to provide the distal portion with sufficient column strength for vaginal insertion. Concurrently, the pressure is also at a level that allows the tampon to be securely held within the accommodation space during tampon applicator manipulation. The proximal portion of the chamber is designed so that the overall volume of air within the chamber is at a level sufficient to allow complete tampon extrusion when the proximal portion is squeezed between the user's fingers.

In use, the tampon applicator invention is unwrapped from its packaging, grasped with the fingers at its proximal end, and guided to the vaginal opening. The distal tip of the applicator is inserted into the vagina and advanced until the larger proximal portion of the applicator meets the vaginal lips. The proximal portion is then squeezed between the user's thumb and fingers to increase the pressure within the applicator chamber and initiate eversion of the inverted distal tip. Continued squeezing force causes the inverted tip to gradually unfold, opening up the vaginal canal adjacent the chamber's distal tip and advancing the tampon upward. The user compresses the proximal portion of the chamber until the thumb and fingers meet. At this point, the tampon is fully unsheathed and it is in its proper position within the vagina.

The tampon applicator can be modified to include a variety of design features that enhance its utility. For example, the annular fold at the distal tip can be configured to provide a rounded profile to aid vaginal introduction. This can be achieved by shaping and arrangement of the distal tip fold or by providing a shaped cap (for example with petalous projections) that allows the tampon to pass through it during delivery from the applicator. This cap can be attached either externally or internally to the chamber.

The applicator can also be provided with features and/or surface treatments to improve insertion and handling. The tampon applicator may be made of a soft feeling material to enhance comfort during insertion. The chamber can be made of a low-friction material or coated with a lubricant or hydrophilic treatment to facilitate vaginal access and minimize trauma. Gripping features or surface treatments may be used to make the proximal portion easier to hold on to during applicator insertion. A flange or bump can be incorporated into the distal portion to act as a stopping point during vaginal insertion.

Features to ensure proper tampon string deployment can also be integrated into the design of the invention. The proximal end of the tampon string may be lightly attached to the end of the evaginated portion so that the string is withdrawn to the outside of the vagina as the applicator is withdrawn, and then the string is released from the applicator. In another configuration, the string may be routed along the outside of the distal portion of the chamber, allowing string access throughout tampon delivery. In this design, the tampon string can run along the surface of the distal portion of the chamber or housed in a channel or sleeve running externally along the distal portion's length. In another design, the string can be housed within a secondary invagination of the receptacle that holds the tampon. The secondary invagination extends into the proximal portion of the chamber so that the string can be visualized if the chamber is made from a clear material. Additionally, an internal stop can be incorporated into the extension member that limits the level of distal portion evagination that can occur, facilitating proper tampon placement. All these designs would be configured to ensure that the applicator can be withdrawn from the vagina without accidental tensioning of the tampon string.

The tampon applicator can be designed to maximize its compactness and disposability. The proximal portion shape and extrusion length of the distal portion can be configured so that the applicator is compact, discrete, and easily handled. An air release feature may be provided to allow complete deflation of the applicator after deployment to decrease discard volume. Furthermore, the tampon applicator may be packaged to further enhance its functionality and cost-effectiveness. For example, the applicator may be packaged so that the distal end may “unpeeled” from the wrapper for tampon placement while a proximal portion of the applicator remains attached to the wrapping, so that applicator and packaging can be disposed of together.

Barometric variations affecting chamber pressurization can be dealt with by providing for a range of interior pressures at which deployment can be achieved. Inadvertent pressure loss can be dealt with through proper design and sealing of the chamber as well as chamber material and gas selection.

The applicator may be made from any number of thin films that are capable of maintaining gas pressure, while providing the structural, surface, and disposal qualities that are desired. More than one material may be used to achieve the desired functionality. The materials may be combined by mixing, laminating, bonding, or other combining methods. The applicator can be manufactured using common thin-film chamber-making processes, including, but not limited to, heat-sealing, RF-sealing, welding, bonding, dipping, injection molding, and blow-molding. These materials include: Low Density and Linear Low Density Polyethylene (LDPE and LLDPE), High Density Polyethylene (HDPE), Vinyl, Polyethylene terephthalate (PET), Polypropylene (PP), Polyvinylidene chloride (PVDC), Ethylene vinyl alcohol (EVOH), Polyester, Nylon, Polyurethane, and Ethylene vinyl acetate (EVA), among others.

Additionally, the applicator may be made from a material that is biodegradable or water soluble for environmentally-friendly disposal. The applicator would be made from a material that is structurally stable during use, but when entered into the waste disposal system either via a toilet sewage system or municipal landfill, the applicator would substantially disintegrate in a timely fashion. Suitable biodegradable materials include thermoplastic starch polymers, polyvinyl alcohols, polylactic acid, cellulose-based polymers, polyoxyethelene, and the like.

### Particular Embodiments

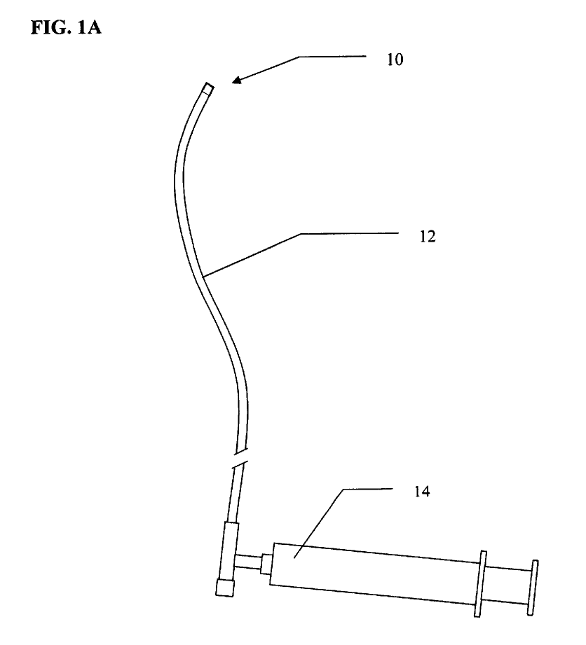

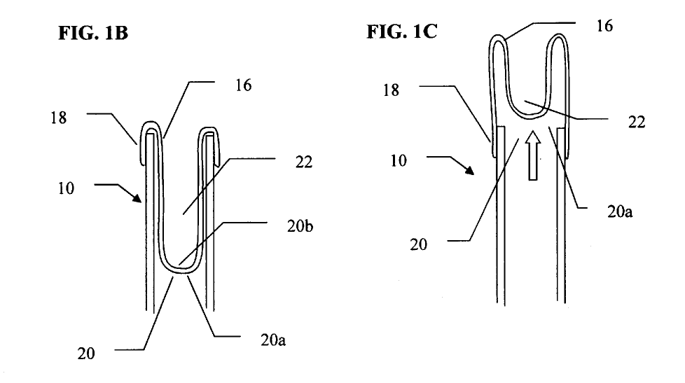

One embodiment of a delivery device 10 is illustrated in FIGS. 1A, 1B, 1C and 2A, 2B and 2C. Delivery device 10 is secured to the distal tip of a flexible catheter 12 that is of sufficient length and rigidity to extend from outside the body to a target location within the body. Except for the presence of delivery device 10, catheter 12 is of a conventional design that is capable of reaching target locations such as into a blood vessel (including the neurovasculature or coronary vessels), a chamber of the heart, the gall bladder, into the urethra or urinary bladder, into the respiratory tract (for example into the pharynx or one of its divisions such as the nasopharynx), into the or into the gastrointestinal tract (such as the esophagus, stomach, intestine, colon or rectum). Catheter 12 may be single-lumen or multi-lumen, for example co-axial or side-by-side lumens. A proximal end of catheter 12 is shown connected to syringe 14, which is a conventional pressurization device for introducing pressure into a lumen of flexible catheter 12. The distal end of catheter 12 is an open cylindrical tip in the illustrated embodiment, with a flat peripheral face that joins an inner wall and outer wall of the catheter.

FIGS. 1 and 2 illustrate delivery device 10 at the distal tip of catheter 12. Delivery device 10 includes a flexible or elastic diaphragm 16 that is secured to the outer wall of the catheter along a sealed seam 18 (FIGS. 1B and 1C) near the open tip, with the diaphragm folded over the open tip of catheter 12 and invaginating into the open tip. Catheter 12 is made of a different, less flexible material than diaphragm 16. The illustrated diaphragm 16 is elastic and generally cylindrical with a rounded end 20, and when invaginated into device 10 it forms a receptacle 22 having a proximal face 20a and a distal face 20b. The dimensions and flexibility of diaphragm 16 permit it to invaginate almost entirely within the distal tip of catheter 12 (FIG. 1B), move toward a mid-evaginated condition (FIG. 1C) in which the receptacle is invaginated only within surrounding portions of the diaphragm, to a fully evaginates condition (not shown) in which the walls of the diaphragm do not overlap as they do in FIG. 1C.

The diaphragm is, for example, similar to a conventional condom, which is made of latex or polyurethane, but other suitable materials include polypropylene. The cylindrical body of diaphragm 16 is of sufficient diameter and length to accommodate an item 24 (FIGS. 2A-2C) that is to be delivered from device 10. The illustrated item 24 is an absorbent member that is generally cylindrical in shape and conforms to the generally cylindrical receptacle formed by invaginated diaphragm 16.

Item 24 can take a variety of forms and shapes. It is generally a solid member, such as a tampon, suppository or other solid or semi-solid pharmaceutical composition (such as a tablet or capsule), coil, stent, prosthetic heart valve, occlusion device or plug, or nasal packing. The illustrated substantially cylindrical receptacle formed by invaginated diaphragm 16 is suitable for holding and delivering items having a variety of shapes, and item 24 need not conform to the shape of the receptacle. However, the shape of the receptacle can be varied to accommodate different items 24.

FIGS. 1B and 1C illustrate the deployment of diaphragm 16 from delivery device 10. An item 24 is not shown in receptacle 22, but would be present in normal operation of device 10. FIG. 1B shows the fully invaginated position of diaphragm 16. When pressure in the lumen of catheter 12 is increased, the pressure acts against proximal face 20a of diaphragm 16. This pressure moves the end 20 of receptacle 22 in the direction of the open distal end of catheter 12, which in turn begins to evaginate diaphragm 16 out of the open end of the catheter, as shown in FIG. 2C. The evaginating diaphragm 16 helps form an ejection pathway in the body as the walls of the diaphragm extend beyond the open tip of the catheter. This function of the walls of the diaphragm is illustrated in FIG. 1C which shows the generally M-shaped cross-sectional configuration of the partially evaginated diaphragm 16. As the leading edge of the evaginating diaphragm moves forward ahead of receptacle 22, those leading edges of the diaphragm help gently push apart the tissue around the evaginating diaphragm. In particular embodiments, the item to be delivered from device 10 has an axial length less than the axial length of receptacle 22, so that the walls of the evaginating diaphragm precede the emergence of the item from receptacle 22.

Use of the delivery device is more fully illustrated in FIGS. 2A, 2B and 2C. The distal tip of device 10 is advanced to a target site in the body of a subject where item 24 is to be delivered. The catheter can be introduced endoscopically with or without a guide wire and positioning of the catheter in the body can be confirmed using conventional imaging technology. Pressure is introduced through catheter 12 by advancing the plunger of syringe 14 (FIG. 1A) to introduce a fluid (such as air or liquid) from the syringe through a lumen of the catheter. The fluid communicates with proximal face 20a of diaphragm 16, which forces the diaphragm toward the open distal end of catheter 12. As the diaphragm moves distally, it begins to eject item 24 (FIG. 2B). At least during the initial stages of ejection, the walls of the diaphragm move ahead of the item to form a protective pathway along which the item is to be delivered. The unfurling diaphragm therefore can assist with spreading tissue ahead of item 24 to facilitate its delivery and placement in the body.

FIGS. 2D, 2E, and 2F illustrate an alternative embodiment of the catheter-based delivery system in which a delivery device 30 is situated within the distal end of a catheter 32 having a side port 34 through which catheter 32 travels over a guidewire 42. A generally cylindrically shaped diaphragm 36 that conforms to the inner walls of catheter 32 is sealed to an internal wall of catheter 32 at a seam 38 which is located slightly proximal to side port 34. Operation of this embodiment is similar to that illustrated in FIGS. 2A-2C. First, catheter 32 is advanced along guidewire 42 until catheter 32 moves past the distal tip of guidewire 42, leaving the catheter's distal tip open (FIG. 2E). Pressure in catheter 32 is increased to apply pressure to the proximal face of diaphragm 36 to advance item 40 distally out the open end of the catheter. Although a monorail type catheter is illustrated in FIGS. 2D, E, and F, a catheter with a dedicated guidewire channel or other guidewire tracking feature can also be used.

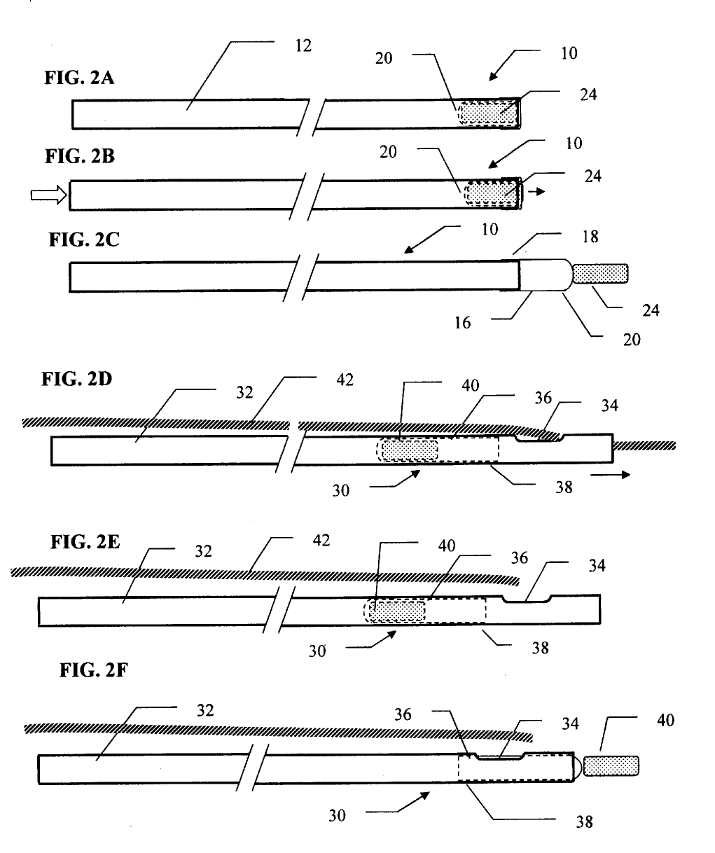

FIGS. 3A, 3B and 3C are similar to FIGS. 2A, 2B and 2C, and illustrate a delivery device 50 at the distal tip of a catheter 52. As in the previous embodiments, delivery device 50 includes an invaginated elastic diaphragm 54 that evaginates to deliver an item 56 from the distal tip of catheter 52. However, in this embodiment item 56 is reversibly attached or sealed to the distal face of diaphragm 54. The seal can take a variety of forms, such as a glue that is sufficiently adhesive to hold item 56 securely against diaphragm 54 when the diaphragm is in the fully invaginated position illustrated in FIG. 3A. However, the dried adhesive is sufficiently frangible that it cracks and loses its ability to hold item 56 to diaphragm 54 as the diaphragm moves (FIG. 3B) and fully evaginates (FIG. 3C). Hence item 56 is held securely in catheter 52 as it is advanced to the target location, but once the diaphragm is mobilized to deliver item 56 the item is easily deployed.

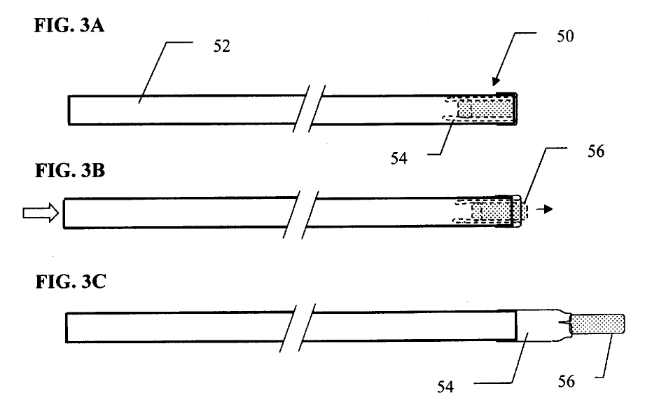

FIGS. 4A, 4B and 4C illustrate an embodiment of the device that forms a self-contained chamber, and is suitable for use as a tampon applicator. The device 60 is an elongated, generally cylindrical, substantially fusiform (rounded or hemispherical at both ends), flexible, gas-impermeable member 62 (FIG. 4C) that is partially pressurized. The walls (or at least portions of the walls) of device 60 enclose a chamber 69 in which the pressure is increased by compressing a portion of the walls of device 60. The pressure within member 62 is sufficiently low that a distal rounded end 64 of member 62 can be invaginated along a longitudinal axis L (FIG. 4A) toward the proximal rounded end 66 of member 62 to form a receptacle 68 (FIGS. 4A and 4B). The invaginated distal portion of device 60 thereby forms the flexible diaphragm that projects into a closed, substantially sealed internal chamber 69 of device 60. In this embodiment, the flexible diaphragm is of the same material as the walls of device 60, so that the entire device (including the diaphragm) has substantially the same flexibility. However, in other embodiments the diaphragm portion of device 60 can be made of a material having different flexibility (for example more flexibility) as long as the walls of chamber 69 are sufficiently flexible to be compressed to raise fluid pressure within the device and induce evagination of the diaphragm.

Device 60 can be assembled by starting with an elongated, unitary, one-piece flexible tube that does not yet have a portion inverted into the tube, as shown in FIG. 4C. The contents of the flexible tube form a sealed chamber defined by a continuous wall. A hemispherical distal end portion of the tube is pushed in into the tube toward the proximal end of the tube to form receptacle 68. The item 70 to be delivered can be used to invaginate the distal tip of the tube into itself, in which case the item is inserted into receptacle 68 as the receptacle forms. Alternatively, receptacle 68 can be formed by indenting the distal end of the tube with another object (such as a finger or pushing rod), and item 70 is subsequently placed in receptacle 68. After the indentation is performed, the device is of a convenient size to be held by hand, for example being less than 12 inches in length. It has a width that is sufficient to hold the item (such as a tampon) within receptacle 68, but not so wide as to be uncomfortable in use. In certain disclosed embodiments, the external diameter of the distal end of the device is less than about 1 or 1.5 inch.

The flexible diaphragm can move from the invaginated to the evaginated position to deliver the item from the receptacle as the volume of the receptacle is progressively reduced and then eliminated. An item to be delivered, such as tampon 70, is placed in receptacle 68 (FIG. 4A) where it is fully contained within the flexible diaphragm and does not extend even in-part outside of receptacle 68. In the illustrated embodiment, receptacle 68 has an axial length (along axis L) and inner diameter (perpendicular to axis L) that is only slightly greater than the axial length and diameter of tampon 70 so that tampon 70 fits snugly within receptacle 68. In the illustrated embodiment, the axial length of receptacle 68 is about half, or less than half, of the axial length of device 60 when the distal portion of device 60 is invaginated within itself to form receptacle 68.

In operation, a user (not shown) grasps the proximal portion 66 of device 60 which serves as a handgrip 72 (FIG. 4A), and inserts the distal end of device 60 into a body cavity where the item is to be delivered. In the illustrated embodiment, the distal portion of the device 60 shown in FIG. 4A is inserted through the introitus into the vagina (not shown) by grasping handgrip 72 without squeezing it sufficiently to pressurize the contents of its internal chamber beyond a delivery pressurization threshold. The distal portion of device 60 is advanced into the vagina to a desired penetration, and the user then firmly directly compresses handgrip 72 with the hand to manually increase pressure within chamber 69 (as illustrated by the multiple arrows within chamber 69 in FIG. 4B). As pressure on handgrip 72 increases beyond a delivery threshold, pressure is exerted against the internal face of the invaginated end of the device as well as against the peripheral lip 74 that externally surrounds receptacle 68. The diameter of receptacle 68 may also slightly decrease as the pressure in chamber 69 increases, but the tampon 70 within the receptacle prevents radial collapse of the receptacle. Instead, the radial compression of receptacle 68 exerts a stabilizing effect on the item in the receptacle to stabilize the item as it begins to move longitudinally out of device 60.

The invaginated end of device 60 begins to evaginate (as illustrated in FIG. 4B) so that the inverted distal portion begins to move distally and tampon 70 is also forced distally as the length and volume of receptacle 68 lessens. Peripheral lip 74 also moves distally to assist with spreading adjacent tissue and holding it apart as the tampon is introduced into the vagina. Direct manual pressure is exerted on handgrip 72 until the invaginated end of device 60 is completely or substantially completely evaginated (FIG. 4C), at which time device 60 resumes its original tubular or fusiform configuration and receptacle 68 is completely or substantially completely eliminated. Tampon 70 is delivered from the device to its target location, and device 60 is then withdrawn from the body.

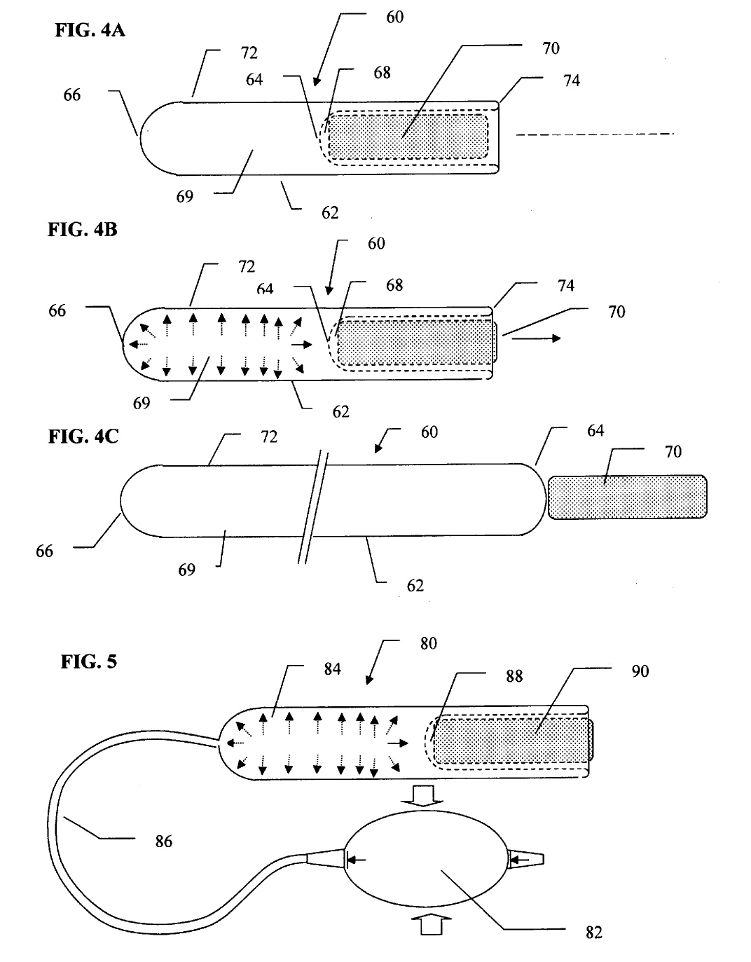

FIG. 5 illustrates a device 80 which is similar to device 60, having the same materials and construction, except it is not a self-contained, pressurized, sealed chamber. Instead, device 80 communicates with an external pressurization device in the form of a compressible, elastic pressurization bulb 82. In other embodiments, bulb 82 could be substituted with a syringe, such as that illustrated in FIG. 1A. The bulb 82 is in fluid communication with internal chamber 84 of device 80 through a flexible pressurization tube 86. Pressure in chamber 84 can be selectively adjusted by compression of bulb 82, and the pressure maintained by selectively closing tube 86 to fluid flow (for example by closing a valve between bulb 82 and chamber 84). In this manner, the rigidity of device 80 can be selectively controlled, for example to sufficiently rigidify it prior to insertion into the body that it retains sufficient column strength for ease of insertion. An initial partial pressurization step can also cause the walls of the receptacle 88 to firmly conform to the item within the receptacle (in this case, tampon 90) which helps stabilize item as it is delivered. Stabilization of the item is particularly advantageous for delivery of an elongated item, such as a tampon. Once device 80 has been introduced through a body orifice, such as the introitus to the vagina or through the nares to the nasopharynx, the absorbent item is deployed from receptacle 88 by additional compression of bulb 82 to evaginate the receptacle so its contents are delivered to the target location.

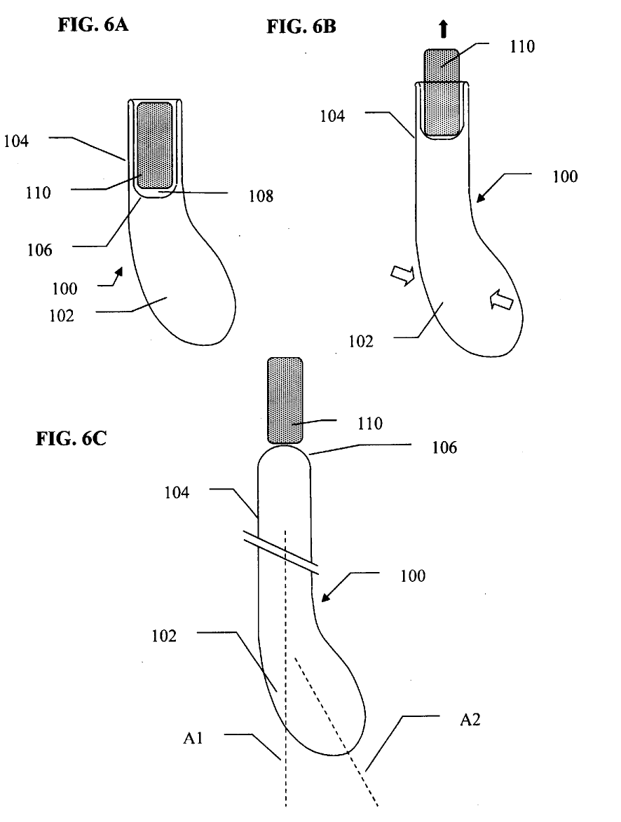

Another embodiment of the applicator is illustrated in FIGS. 6A, 6B and 6C. This applicator is another self-contained hand-held device, similar to that shown in FIGS. 4 and 5. However, instead of being a substantially cylindrical device that extends substantially symmetrically along a longitudinal axis L of the device (FIG. 4A), it is configured for more ergonomic use. As shown in FIG. 6C, the illustrated device 100 includes a bulbous proximal portion 102 and a substantially cylindrical distal portion 104 with a hemispherical tip 106. Portions 102, 104 are angled to one another so that proximal portion 102 forms an angled hand grip that improves the ergonomics of the device in use. The angle between the portions of device 100 are illustrated by axis A1 (which is an axis of symmetry of bulbous proximal portion 102) and axis A2 (which is an axis of symmetry of cylindrical distal portion 104). Portions 102, 104 can be at any convenient angle to one another, such as the illustrated angle of about 30 degrees, however in other examples the angle is between 15-90 degrees, for example between 15-45, 15-30, 30-45 or 30-90 degrees.

To load device 100, the device is provided in its fully extended form shown in FIG. 6C. Hemispherical tip 106 of portion 104 is pushed into itself (for example with a finger or push rod or the item to be loaded in the device) to invert tip 106 into portion 104 and form receptacle 108 so that the exterior of distal portion 104 (FIG. 6C) forms the interior wall of receptacle 108. In the illustrated embodiment, a tampon 110 is aligned with its longitudinal axis generally along axis A2, the end of the tampon 110 is pushed into cap 106 and used to invaginate portion 104 to form receptacle 108.

In use, handle grip proximal portion 102 is grasped by the user and manipulated to guide distal portion 104 to the body opening through which an item is to be delivered. The distal portion 104 is advanced into the body opening until the angled bulbous portion of the device reaches the body orifice. The enlarged diameter and different axis of proximal portion 102 is an indication that optimal insertion of device 100 has been achieved, hence the configuration of the device is used an in indicator. The handle grip proximal portion 102 is then squeezed to a sufficient extent that pressure within device 100 increases to evaginate tip 106 and expel tampon 110 from the receptacle.

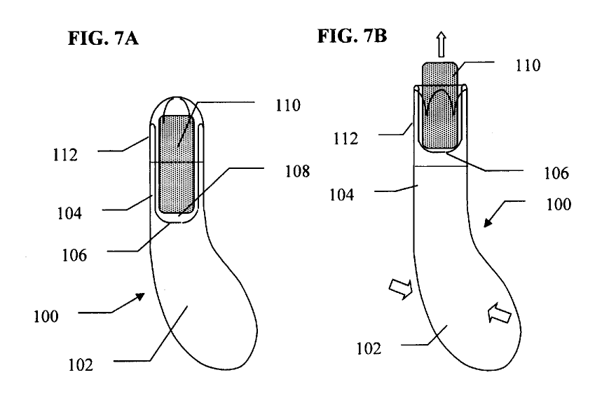

FIGS. 7A and 7B illustrate a device similar to that shown in FIGS. 6A, 6B and 6C such that similar parts are referred to with like reference numbers. However, an external plastic cap 112 with petalous projections is provided on the distal end of device 100. The cap can be applied after the device 100 is loaded with the item to be dispensed (such as tampon 110) to help maintain the item in chamber 108 during use of the device. When the handgrip is compressed to increase pressure within device 100 and deliver item 110 from receptacle 108, pressure is increased on the internal face of receptacle to advance tampon 110 through the petalous projections to eject tampon 110.

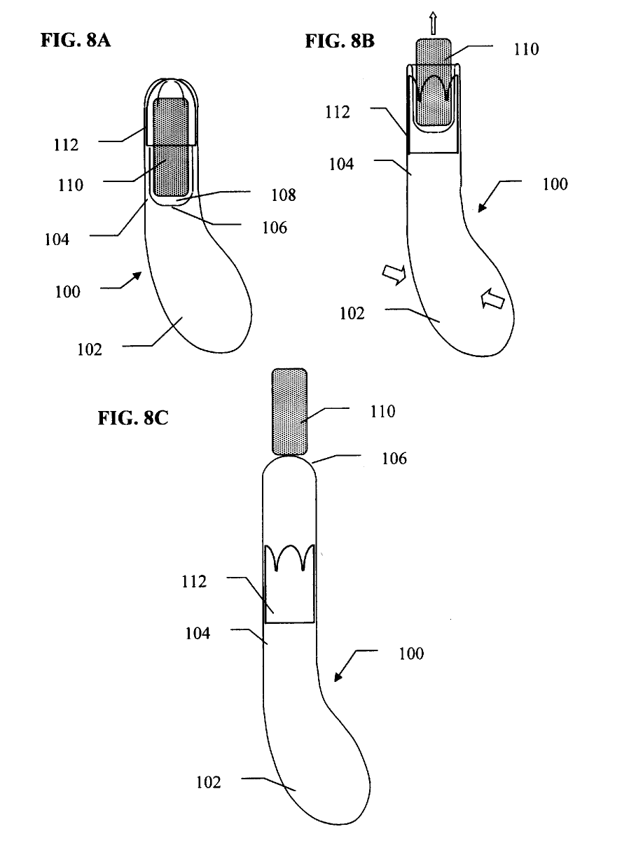

FIGS. 8A, 8B and 8C also illustrate a device similar to that shown in FIGS. 6A, 6B and 6C and FIGS. 7A and 7B such that similar parts are referred to with like reference numbers. However, plastic cap 112 with petalous projections is situated within device 110. When device 100 is loaded with tampon 110 (or other formed object to be delivered), distal tip 106 of the device and tampon 110 are invaginated from the position shown in FIG. 8C through the petalous projections until distal portion 104 and tampon 110 pass completely through the petalous projections. As shown in FIG. 8A, receptacle 108 and tampon 110 are both internal to cap 112. Hand grip proximal portion 102 is them compressed as previously described to deliver tampon 110 through the cap.

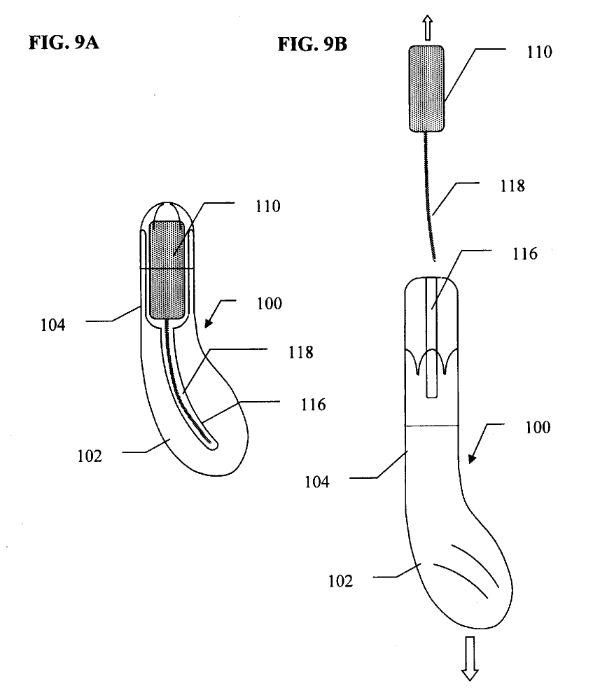

FIGS. 9A and 9B illustrate an embodiment of the device similar to that shown in FIGS. 7A and 7B, hence similar parts have been given like reference numbers. However, in this embodiment, the distal portion 104 of device 100 has an elongated cylindrical receptacle 116 extending along the longitudinal axis of portion 104 along almost the entire length of portion 104. Receptacle 116 communicates with the exterior tip 106 of hemispherical tip 106, such that when tip 106 is invaginated into device 100 (FIG. 9A), receptacle 116 will form a secondary invagination that extends from tip 106 into proximal portion 102.

This embodiment of the device is used to provide a holder for a retrieval string 118 that is attached to tampon 110. As shown in FIG. 9B, string 118 is inserted into receptacle 116 when distal portion 103 is in its fully extended form. As tampon 110 is further pressed against tip 106 to invaginate tip 106 into device 100, string 118 is retained in receptacle 116. Once tampon 110 is deployed, device 100 is withdrawn, leaving the proximal end of tampon string behind to remain outside the vaginal opening.

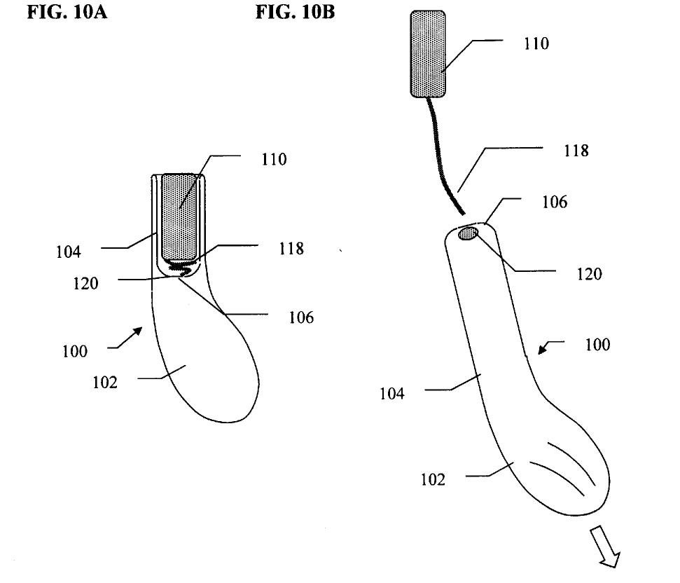

FIGS. 10A and 10B illustrate an embodiment of the device similar to that shown in FIGS. 9A and 9B, hence similar parts have been given like reference numbers. However, in this embodiment, string 118 is lightly attached to tip 106 at attachment feature 120. Attachment feature 120 can consist of a small strip of adhesive material or other means that provides an easily releasable connection between the string end and the device.

This embodiment of the device is used to ensure that the proximal tip of string 118 remains tethered to device 100 as the device is withdrawn. Once tampon 110 is deployed, device 100 is withdrawn. As device 100 is withdrawn, the proximal end of string 118 follows the device out of the vagina. Upon exiting the vagina, string 118 is disconnected from device 100 by lightly pulling the device away from the vaginal opening.

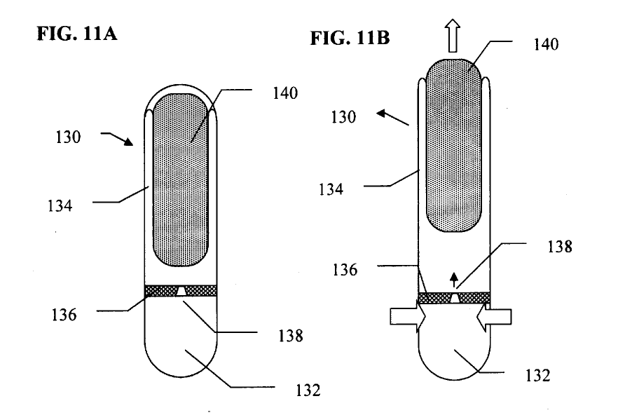

FIGS. 11A and 11B illustrate an embodiment featuring pre-pressurized chamber 132, separated from pre-pressurized distal portion 134 of device 130 by seal 136 that offers increased pressurization capability compared with manually squeezed devices described above. Normally-closed valve 138 allows fluid communication between chamber 132 and distal portion 134. In use, device 130 is partially inserted into the vagina. Tampon 140 is deployed by opening valve 138 to release pressurized gas from chamber 132 causing eversion of distal portion 134 and ejection of tampon 140 into the vagina. FIG. 11B illustrates partial eversion of distal portion 134 and partial ejection of tampon 140 from chamber 132. Continued introduction of pressured air from chamber 132 to 134 continues the eversion of distal portion 134 until tampon 140 is complete ejected from the device.

Valve 138 may be a pressure relief valve in which flow may be activated by squeezing chamber 132 above a certain pressure. Valve 138 can consist of a lightly-sealed portion of seal 136 that is ruptured when chamber 132 is squeezed. Alternatively, valve 138 may be a manually-operated valve, such that fingertip manipulation on or around the valve itself causes it to open.

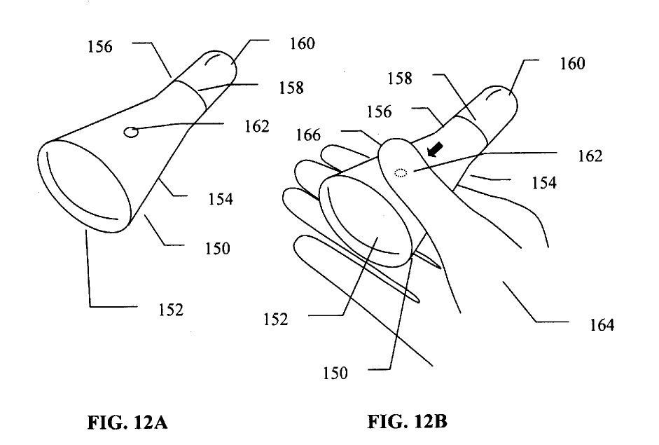

FIGS. 12A and 12B illustrate a frustoconical shaped resilient dispenser body 150 having a slightly convex base 152 and side wall 154 that tapers to a uniform diameter cylindrical neck 156 having a top circular border 158. Neck 156 forms a more elongated cylinder than is shown in FIG. 12, but the elongated neck is invaginated (not shown) into the interior of the dispenser to provide a receptacle for an item that is to be dispensed, such as a tampon 160. In particular embodiments the item to be dispensed is generally cylindrical, and of similar dimensions to the receptacle formed by the invaginated portion of neck 156. An opening 162 in sidewall 154 permits air to flow between the interior and exterior of dispenser body 150.

In the embodiment shown in FIG. 12A, a one-way valve beneath inlet 162 governs the flow of air into and out of resilient dispenser body 150. When body 150 is compressed to evert the invaginated neck portion of the device and dispense tampon 160 from the device, the valve does not open until a sufficient internal pressure has been achieved within body 150 to evert the neck portion. Once that sufficient internal pressure level has been reached, the valve permits air to flow out of the body if further compression is desired. When pressure is no longer applied to it, resilient body 154 then expands to the fully expanded condition shown in FIG. 12A as the valve in inlet 162 permits air to flow freely into body 154.

In another embodiment shown in FIG. 12B, a valve is not placed in opening 162 but airflow is instead regulated by selectively sealing the opening, for example by placing a portion of a digit (such as the thumb) over the opening. As illustrated in FIG. 12B, resilient body 150 with tampon 160 loaded therewithin is held by the hand 164 of a user, with the fully expanded body 150 cradled between the fingers and the thumb 166 of the hand. The tip of tampon 160 is then inserted into the vagina while at least a portion of the enlarged diameter base remains external to the body where it provides an easily grasped portion of the device that inhibits over-insertion. Hole 162 is ideally located in a portion of the device that is of a sufficiently large diameter that it is not inserted into the body.

When it is desired to deliver tampon 160 from body 150, thumb 166 is placed over opening 162 to inhibit escape of air inside body 150 through opening 162. Body 150 is them compressed between the fingers and thumb of the operator's hand to increase pressure within body 150 and evert invaginated neck 156 to deliver tampon 160 to a desired location, such as the vagina.

In alternative embodiments (not shown), body 150 is filled with a resilient material (such as an open cell foam) that normally retains body 150 in an expanded condition, such as that shown in FIGS. 12A and 12B. The open cell foam can be compressed to increase pressure within body 150 and expel tampon 160 from neck 156 by evaginating the inverted neck portion.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A device for delivering an item, the device comprising:
a self-contained chamber comprising flexible compressible walls that substantially surround and seal the chamber, wherein the chamber contains a sufficient amount of a gas to provide a pre-deployment pressure within the chamber, and wherein pressure in the chamber is increasable to a deployment pressure by compressing a portion of the flexible walls of the chamber;
wherein a portion of the flexible walls forms a flexible diaphragm that is invaginated into the chamber at the pre-deployment pressure to form a receptacle for holding an item for delivery from the receptacle, and the invaginated diaphragm is capable of being evaginated for delivering the item from the receptacle when the pre-deployment pressure is increased to the deployment pressure by compressing the portion of the flexible walls of the chamber.

2. The device of claim 1, further comprising the item for delivery, wherein the chamber is at the pre-deployment pressure and the item is contained at least partially within the receptacle to inhibit radial collapse of the receptacle.

3. The device of claim 1, wherein the chamber comprises a distal portion that forms a delivery portion from which the item is to be delivered from the device and a proximal portion that forms a hand grip for the device, wherein the receptacle invaginates into the distal portion of the chamber at the pre-deployment pressure.

4. The device of claim 1, wherein the device is a one-piece elongated unit comprising a single flexible wall around a sealed interior, wherein the single flexible wall forms both the chamber and the flexible diaphragm.

5. The device of claim 3 wherein the hand grip is wider than the delivery portion of the device, and a portion of the flexible walls of the chamber is compressible by squeezing the hand grip which increases pressure in the chamber from the pre-deployment pressure to the deployment pressure.

6. The device of claim 5, wherein the delivery portion of the device is dimensioned to be insertable into an external orifice of the human body, and the hand grip is sufficiently wide not to be insertable into the external orifice.

7. The device of claim 6, wherein the external orifice is the vagina.

8. The device of claim 1, further comprising a displaceable cap over the receptacle for holding the item in the receptacle prior to delivery.

9. The device of claim 3, wherein the proximal portion of the device is bulbous in shape, and the distal portion of the device is fusiform in shape.

10. The device of claim 3, wherein the delivery portion and the hand grip are inclined at an angle to one another whereby the hand grip provides an angled handgrip to be grasped by a user for inserting the delivery portion into an orifice of a body.

11. The device of claim 10, further comprising a secondary invagination from the receptacle into chamber for accepting a retrieval string attached to the item.

12. The device of claim 1, wherein the self-contained chamber comprises a first sub-chamber and a second sub-chamber, wherein a pressure in the first sub-chamber is greater than a pressure in the second sub-chamber such that selective release of pressure from the first sub-chamber to the second sub-chamber evaginates the flexible membrane.

13. A device for delivering an item, comprising:
a self-contained chamber comprising a single flexible compressible wall that substantially surrounds and seals the chamber, wherein the chamber contains a sufficient amount of a gas to provide a pre-deployment pressure within the chamber, and wherein pressure in the chamber is increasable to a deployment pressure by compressing a portion of the flexible walls of the chamber;
wherein a portion of the flexible wall forms a flexible diaphragm that is invaginated into the chamber at the pre-deployment pressure to form a receptacle for holding an item for delivery from the receptacle, and the invaginated diaphragm is evaginated for delivering the item from the receptacle when the pre-deployment pressure is increased to the deployment pressure;
wherein the chamber comprises a distal portion that forms a delivery portion from which the item is to be delivered from the device and a proximal portion that is wider than the delivery portion and forms a hand grip for the device, wherein the receptacle invaginates into the distal portion of the chamber at the pre-deployment pressure;
wherein the distal end of the device is dimensioned to be insertable into an external orifice of the human body, and the hand grip is sufficiently wide not to be insertable into the external orfice; and
wherein the delivery portion and the hand grip are inclined at an angle to one another whereby the hand grip provides an angled handgrip to be grasped by a user for inserting the delivery portion into an orifice of a body.

14. The device of claim 13, wherein the item is a tampon and the external orifice of the body is a vaginal opening.

15. A method of delivering an item into a body through an external orifice of the body, the method comprising:
inserting the delivery portion of the device of claim 3, with the item in the receptacle of the device, into an external orifice of a body cavity; and
compressing the flexible walls of the chamber to increase pressure in the chamber to the deployment pressure to evaginate the diaphragm and deliver the item from the receptacle into the body cavity.

16. The method of claim 15, wherein the body cavity is a vagina, the external orifice is the vaginal introitus, and the item is a tampon.

17. The method of claim 15, wherein the hand grip is wider than the delivery portion of the device, and a portion of the flexible walls of the chamber is compressible by squeezing the hand grip which increases pressure in the chamber from the pre-deployment pressure to the deployment pressure, and hand grip is compressed to deliver the item from the delivery portion of the device into the body through the external orifice.

18. The method of claim 17, wherein the delivery portion and the hand grip are inclined at an angle to one another whereby the hand grip provides an angled handgrip to be grasped by a user for inserting the delivery portion into the external orifice of the body, and inserting the delivery portion of the device comprises grasping the hand grip and inserting the delivery portion into the external orifice at an inclined angle to the hand grip.
